(19) United States
(12) Patent Application Publication
(10) Pub. No.: US 2007/0142341 A1
(43) Pub. Date: Jun. 21, 2007

(54) Use of steroid derivatives for the treatment of angiotensin ll related disease e.g. cardiovascular and proliferative disorders

(76) Inventors: John Wilbraham Lester, East Sussex (GB); Gavin Paul Vinson, London (GB)

Correspondence Address: JONES DAY, 222 EAST 41ST ST, NEW YORK, NY, 10017, US

(21) Appl. No.: 10/553,111

(22) PCT Filed: Apr. 16, 2004
(86) PCT No.: PCT/GB04/01663

§ 371 Date: Nov. 6, 2006

(30) Foreign Application Priority Data
Apr. 16, 2003 (GB) 0308857.2

(51) Int. Cl. A61K 31/58

(52) U.S. Cl. 514/172

(57) ABSTRACT

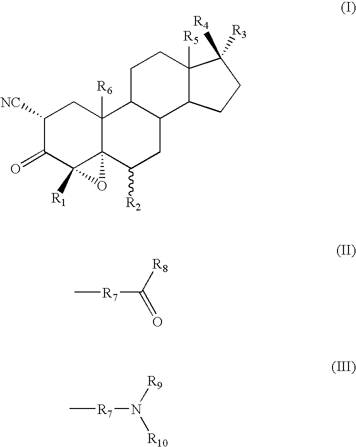

Use of a compound of formula (I) or a 3-enol C_{1 to 4} alkanoate ester thereof in the manufacture of a medicament for the treatment of an angiotensin II related disease in humans and animals wherein R₁, R₂, R₅, R₆ are the same or different and each is hydrogen or C_{1 to 4} alkyl; R₃ is hydrogen, C_{1 to 4} alkyl, C_{2 to 4} alkenyl or C_{2 to 4} alkynyl; R₄ is hydroxyl, C_{1 to 4} alkanoyloxy, a group of formula (II) or (III) wherein R₇ is (CH₂)ₙ, where n is an integer of from 0 to 4, R₈ is hydrogen, C_{1 to 4} alkyl, hydroxy or NH₂ and R₉ and R₁₀ are the same or different and each is hydrogen or C_{1 to 4} alkyl; or R₃ and R₄ together are oxo, ethylenedioxy or propylenedioxy.

The present invention relates to the use of pharmaceutical compositions comprising trilostane or a related compound as active ingredient in the treatment of angiotensin II related disease, in particular angiotensin II related cardiovascular disease.

Regulation of angiotensin II levels in the body is an important factor in both preventing cardiovascular disease and alleviating its effects. Angiotensin II produces several actions in the body, some of which lead directly to cardiovascular disease; others lead to the production of different hormones, for example mineralocorticoids such as aldosterone, which in turn cause the disease. The present invention relates to the use of trilostane, or related compounds, which have been found to modulate the action of angiotensin II receptors in the body, for treating cardiovascular disease.

Cardiovascular function is under the influence of a complex system of inter-related and inter-acting hormones that are released into the systemic circulation by various organs in the body. The renin-angiotensin-aldosterone (RAAS) system is one of the major hormone groups involved.

In this system the kidney secretes the proteolytic enzyme renin which acts on angiotensinogen, a plasma protein, splitting off a fragment containing 10 amino acids called angiotensin I. Angiotensin I is cleaved by a peptidase enzyme secreted by blood vessels, called angiotensin converting enzyme (ACE), producing angiotensin II, which contains 8 amino acids. Angiotensin II (Ang II) has a range of actions in the body, including constriction of the walls of arterioles, closing down capillary beds, stimulation of smooth muscle cell growth in the wall of arterioles thereby causing constriction, stimulation of the tubules in the kidney to reabsorb sodium ions and stimulation of the adrenal cortex to release aldosterone.

Aldosterone causes the kidneys to reclaim still more sodium and, thus, water, and increases the strength of the heartbeat and stimulates the pituitary to release the antidiuretic hormone (ADH, also known as arginine vasopressin).

In addition to the systemic role it is now believed that these hormones are also produced in the tissues of certain organs and act locally as well as at the systemic level. Although local renin-angiotensin systems had been described as functionally distinct systems, recent experimental studies have suggested an association between hyperactivity of these local renin-angiotensin systems and cardiovascular dysfunction. For example, some studies indicate that the human cardiac renin-angiotensin system may be activated in heart disease. Furthermore, polymorphisms in genes coding for the renin-angiotensin system seem associated with hypertension and left ventricular hypertrophy (Clin Exp Hypertens 1995 April; 17(3):441-68).

The existence of a local cardiovascular renin-angiotensin system (RAS) is often invoked to explain the long-term beneficial effects of RAS inhibitors in cardiovascular disease. However, it may be that not all the components of the RAS are synthesized in situ, so that local angiotensin II formation may occur independently of the circulating RAS. Local angiotensin formation in heart and vessel wall does occur, but may depend, at least under normal circumstances, on the uptake of renal renin from the circulation. Tissues may regulate their local angiotensin concentrations by varying the number of renin receptors and/or renin-binding proteins, the ACE level, the amount of metabolizing enzymes and the angiotensin receptor density. Binding of renin to cardiac vascular membranes may therefore be part of a mechanism by which renin is taken up from plasma.

In heart failure, aldosterone has been implicated in the formation of reactive interstitial fibrosis, a maladaptation that contributes to left ventricular remodeling. A recent study (Endocrinology 2002 December; 143(12):4828-36) described the role of aldosterone in myocardial injury in a rat model. Angiotensin caused injury to the heart, including arterial fibrinoid necrosis, perivascular inflammation (primarily macrophages), and focal infarctions. Vascular lesions were associated with expression of the inflammatory mediators cyclooxygenase 2 (COX-2) and osteopontin in the media of coronary arteries. Myocardial injury, COX-2, and osteopontin expression were markedly attenuated by treatment with eplerenone (a new aldosterone blocker). The study concluded that aldosterone plays a major role in Ang II-induced vascular inflammation in the heart and implicated COX-2 and osteopontin as potential mediators of the damage. Somewhat similar findings were made in a study of the effects of eplerenone in dogs with chronic heart failure (Circulation 2002 Dec. 3; 106 (23):2967-72). In this study heart failure was produced in dogs by intracoronary microembolizations that were discontinued when left ventricular (LV) ejection fraction (EF) was between 30% and 40%. In control dogs, LV end-diastolic and end-systolic volume increased significantly. In contrast, end-diastolic volume, end-systolic volume, and EF remained unchanged during the 3 months of treatment with eplerenone. LV end-diastolic wall stress increased significantly in control dogs but decreased significantly in eplerenone-treated dogs. Compared with control, eplerenone was associated with a 28% reduction in cardiomyocyte cross-sectional area, a 37% reduction of volume fraction of reactive interstitial fibrosis, and a 34% reduction of volume fraction of replacement fibrosis. The study concluded that long-term therapy with eplerenone prevented progressive LV dysfunction and attenuated LV remodeling in dogs with chronic heart failure.

ACE inhibitors, in addition to their proven role in the treatment of hypertension, are used also for the treatment of cardiac failure. Clinical trials have shown that these agents, in addition to improving cardiac function, reduce mortality in heart failure. One therapeutic mechanism by which they treat heart failure is believed to be the reduction of circulating angiotensin II and aldosterone. However, the Renin-Angiotensin-Aldosterone axis (RAAS) is not uniformly suppressed during therapy for heart failure. This effect has been referred to as ‘angiotensin II reactivation’ which may herald clinical deterioration. In a large-scale clinical trial, referred to as the CONSENSUS I trial, correlations were seen between mortality, and angiotensin II and aldosterone. Furthermore, mortality was lower in those with good angiotensin II suppression. Therefore, it has been suggested (Eur J Heart Fail 1999 December;1(4):401-6) that neurohormonal elevation despite adequate treatment may associate with a poorer prognosis.

In the Randomized ALdactone Evaluation Study (RALES), spironolactone, an aldosterone receptor antagonist, significantly reduced mortality in patients with severe congestive heart failure (CHF). Spironolactone was given in addition to ACE inhibitors and its effect was additive to these agents (J Am Coll Cardiol 2002 Nov. 6; 40(9): 1596-601)

Trilostane, (4α,5α-17β)-4,5-epoxy-3,17-dihydroxyandrost-2-ene-2carbonitrile, is described in British Patent Specification No. 1,123.770 and in the U.S. Pat. No. 3,296,295.

GB 2,130,588 relates to an improved method of manufacture for trilostane and related compounds. This method allowed the micronising of the compounds to particles having a mean equivalent sphere volume diameter of from 5 to 12 mm, with at least 95% of the particles having a particle size of less than 50 mm. The greater specificity of particle size improves the bio-availability of trilostane and controls the amount of active metabolite formed, thus improving the clinical response and decreasing variability.

The inventors have surprisingly found that trilostane and related compounds inhibit the proliferative effects of angiotensin II on smooth vascular muscle cells, without necessarily lowering levels of mineralocorticoids, such as aldosterone, in the plasma thereby allowing treatment of proliferative diseases associated with these hormones. It is believed that the inhibition of the proliferative effects of angiotensin II on smooth vascular muscle cells, without necessarily lowering levels of mineralocorticoids, such as aldosterone, in the plasma arises through the reduction of sensitivity of angiotensin II receptors. The reduction of sensitivity of angiotensin II receptors may, for example, result from impaired production of an intracellular signal, such as a calcium signal, and by reducing the expression of angiotensin II type 1 (AT1) receptors.

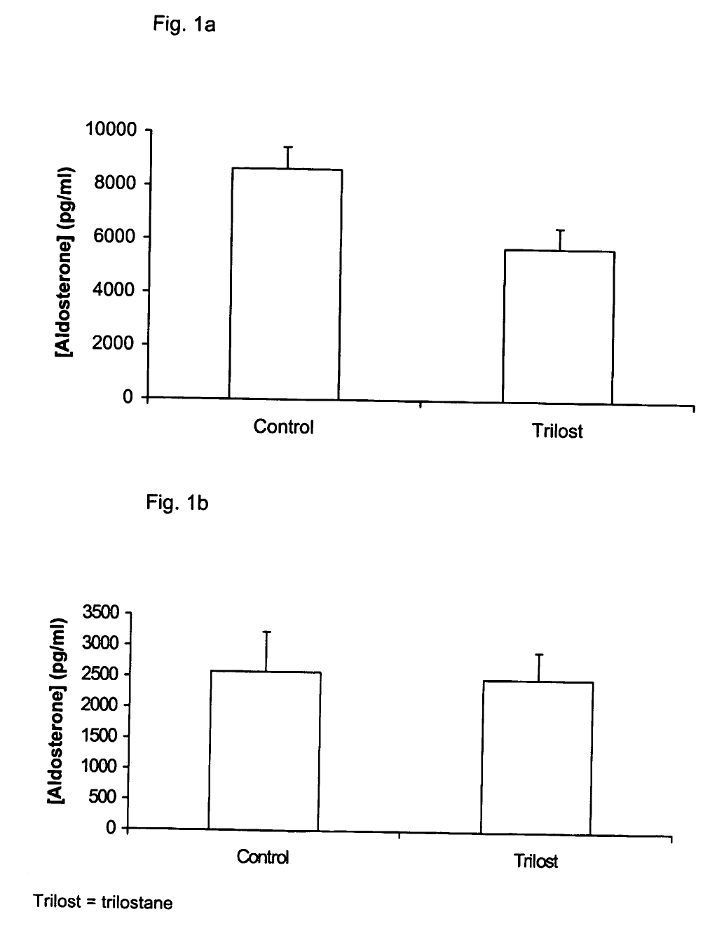

Trilostane has been used in treatments that are aimed at suppressing adrenal steroid secretion. Examples of adrenal steroids include cortisol, aldosterone and corticosterone. In practice, in patients with normally functioning adrenals, circulating adrenal steroids are reduced only at high trilostane dosage levels upwards of 8 to 10 mg/kg/day equivalent, and this is the regime most frequently used (Beardwell et al. 1985, Clin Endocrinol (Oxf), 23, 413-21, Engelhardt and Weber 1994, J Steroid Biochem Mol Biol, 40, 261-7). These data can be reproduced in whole, healthy rats, in which trilostane at 8 mg/kg/day reduces concentrations of aldosterone in circulating plasma (FIG. 1a). This can be shown by testing the levels of circulating adrenal steroids pre- and post-treatment and assessing whether or not the concentrations of adrenal steroids have been reduced. The levels of circulating adrenal steroids in the plasma can be tested by collecting circulating blood from a vein. Plasma is obtained by centrifugation and plasma steroid (for example, cortisol and aldesterone for humans, corticosterone and aldosterone in rats) is assayed using a conventional radioimmunoassay. Suitable corticosterone radioammunoassay kits are available from Amersham Biosciences UK Limited. Suitable aldosterone radioimmunoassay kits are available from Diagnostic Products Corporation.

The present inventors have found that a lower concentration, such as 4 mg/kg/day, does not reduce concentrations of aldosterone in circulating plasma (FIG. 1b). Neither dose (4 mg/kg/day or 8 mg/kg/day) affects circulating corticosterone levels, for which still higher doses are required.

Accordingly, in one embodiment, the present invention relates to the use of trilostane and related compounds in treating angiotensin II related disease in effective doses at levels at which circulating adrenal steroid concentrations are not affected.

One advantage that this regime brings is that side effects of excessive trilostane treatment, namely hypocortisolism and hypoaldosteronism, are avoided, and the concomitant administration of a glucocorticoid such as hydrocortisone (cortisol), dexamethasone or betamethasone, is avoided.

Accordingly the invention provides:

Use of a compound of formula (I) or a 3-enol C_{1 to 4} alkanoate ester thereof in the manufacture of a medicament for the treatment of angiotensin II related disease in humans and animals 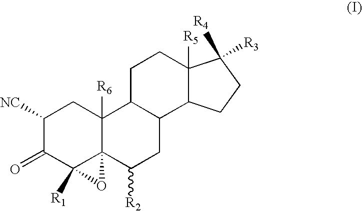
wherein R₁, R₂, R₅, R₆ are the same or different and each is hydrogen or C_{1 to 4} alkyl;
R₃ is hydrogen, C_{1 to 4} alkyl, C_{2 to 4} alkenyl or C_{2 to 4} alkynyl;
R₄ is hydroxyl, C_{1 to 4} alkanoyloxy, a group of formula (II) or (III)

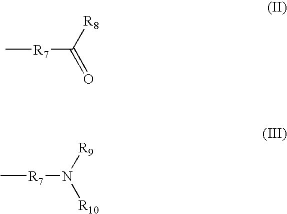

wherein R₇ is (CH₂)ₙ, where n is an integer of from 0 to 4, R₈ is hydrogen, C_{1 to 4} alkyl, hydroxy or NH₂ and R₉ and R₁₀ are the same or different and each is hydrogen or C_{1 to 4} alkyl;

or R₃ and R₄ together are oxo, ethylenedioxy or propylenedioxy;

Use of a compound of formula (I) or a 3-enol C_{1 to 4} alkanoate ester thereof in the manufacture of a medicament for the treatment of angiotensin II related cardiovascular disease in humans and animals 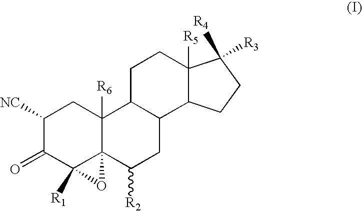
wherein R₁, R₂, R₅, R₆ are the same or different and each is hydrogen or C_{1 to 4} alkyl;
R₃ is hydrogen, C_{1 to 4} alkyl, C_{1 to 4} alkenyl or C_{1 to 4} alkynyl;
R₄ is hydroxyl, C_{1 to 4} alkanoyloxy, a group of formula (II) or (III)

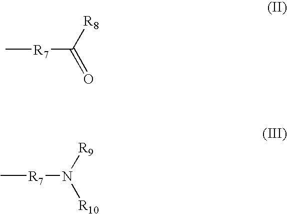

wherein R₇ is (CH₂)ₙ, where n is an integer of from 0 to 4, R₈ is hydrogen, C_{1 to 4} alkyl, hydroxy or NH₂ and R₉ and R₁₀ are the same or different and each is hydrogen or C_{1 to 4} alkyl;

or R₃ and R₄ together are oxo, ethylenedioxy or propylenedioxy;

Use of a compound of formula (I) or a 3-enol C_{1 to 4} alkanoate ester thereof, as defined above, in the manufacture of a medicament for the treatment of an angiotensin II related disease in humans and animals wherein the treatment is carried out in combination with the administration of one or more of:
an Angiotensin Converting Enzyme (ACE) inhibitor;
an angiotensin II receptor blocker;
an aldosterone inhibitor or agent for lowering aldosterone levels or blocking the effects of aldosterone; or
a steroidogenesis inhibitor; and

Use of a compound of formula (I) or a 3-enol C_{1 to 4} alkanoate ester thereof in the manufacture of a medicament for the treatment of an angiotensin II related disease in humans and animals wherein the treatment is carried out in combination with the administration of one or more of:
an Angiotensin Converting Enzyme (ACE) inhibitor;
an angiotensin II receptor blocker; or
an aldosterone inhibitor or agent for lowering aldosterone levels or blocking the effects of aldosterone.

In a preferred embodiment, the present invention relates to the use of a compound of formula (I) or a 3-enol C_{1 to 4} alkanoate ester, as defined above, in the manufacture of a medicament, as defined above, wherein said medicament is administered in an amount of from 0.5 to 4 mg/kg/day.

The present invention further provides:

A medicament comprising:
(a) a compound of formula (I) or a 3-enol C_{1 to 4} alkanoate ester thereof, as defined above; and
(b) one or more of:
   an ACE inhibitor;
   an angiotensin II receptor blocker;
   an aldosterone inhibitor or agent for lowering aldosterone levels or blocking the effects of aldosterone; or
   a steroidogenesis inhibitor;

A medicament comprising:
(a) a compound of formula (I) or a 3-enol C_{1 to 4} alkanoate ester thereof; and
(b) one or more of:
   an ACE inhibitor;
   an angiotensin II receptor blocker; or
   an aldosterone inhibitor or agent for lowering aldosterone levels or blocking the effects of aldosterone;

A method of treating an angiotensin II related disease by administering to a patient having said disease a compound of formula (I) or a 3-enol C_{1 to 4} alkanoate ester thereof, as defined above, in an amount effective to treat said disease;

A method of treating an angiotensin II related cardiovascular disease by administering to a patient having said disease a compound of formula (I) or a 3-enol C_{1 to 4} alkanoate ester thereof in an amount effective to treat said disease;

A method of treating an angiotensin II related disease by administering to a patient having said disease an amount of formula (I) or a 3-enol C_{1 to 4} alkanoate ester thereof, as defined above, and an amount of one or more of:
an ACE inhibitor;
an angiotensin II receptor blocker;
an aldosterone inhibitor or agent for lowering aldosterone levels or blocking the effects of aldosterone; or
a steroidogenesis inhibitor effective to treat said disease; and

A method of treating an angiotensin II related cardiovascular disease by administering to a patient having said disease an amount of formula (I) or a 3-enol C_{1 to 4} alkanoate ester thereof, and an amount of one or more of:
an ACE inhibitor;
an angiotensin II receptor blocker; or
an aldosterone inhibitor or agent for lowering aldosterone levels or blocking the effects of aldosterone
   effective to treat said disease.

Typically, the present methods of treating an angiotensin II related disease, as defined above, comprise administering a compound of formula (I) or a 3-enol C_{1 to 4} alkanoate ester to a patient having said disease in an amount which is non-toxic and effective to treat said disease.

As used herein, a C_{1 to 4} alkyl group or moiety is a straight or branched-chain alkyl group containing from one to four carbon atoms, such as methyl, ethyl, n-propyl, i-propyl, n-butyl and t-butyl. Typically, said alkyl group is unsubstituted. Typically, the C_{1 to 4} alkyl group or moiety is a straight chain alkyl group, such as methyl, ethyl, n-propyl and n-butyl. Preferably, a C_{1 to 4} alkyl group or moiety is methyl.

A C_{1 to 4} alkenyl group is an olefinic group containing from two to four carbon atoms. A C_{2 to 4} alkenyl group is, for example, ethenyl, n-propenyl, i-propenyl, n-butyenyl, i-butenyl, s-butenyl and t-butenyl. An alkenyl group typically contains only one double bond. Typically, said alkenyl group is unsubstituted.

A C_{1 to 4} alkynyl group is a linear or branched alkynyl group containing from two to four carbon atoms. A C_{2 to 4} alkynyl is, for example, ethynyl, n-propynyl or n-butynyl. Typically, an alkynyl group contains only one triple bond. Typically, said alkynyl group is unsubstituted.

A C_{1 to 4} alkanoyloxy group is typically a group of formula RₐC(O)O—, wherein Rₐ is hydrogen or a C_{1 to 3} alkyl group such as methyl, ethyl, n-propyl or i-propyl. Typically, said C_{1 to 3} alkyl group is unsubstituted. Preferably the C_{1 to 3} alkyl group is a straight chain alkyl group, such as methyl, ethyl or n-propyl.

A 3-enol C_{1 to 4} alkanoate ester of a compound of formula (I) has the structure shown in formula (Ia)

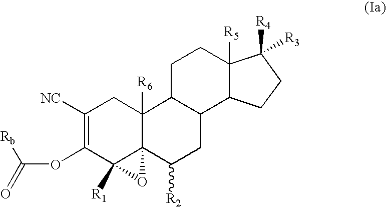

wherein R₁ to R₆ are as defined above and R_{b} is hydrogen or a C_{1 to 3} alkyl group such as methyl, ethyl, n-propyl or i-propyl. Typically, said C_{1 to 3} alkyl group is unsubstituted. Preferably the C_{1 to 3} alkyl group is a straight chain alkyl group, such as methyl, ethyl or n-propyl.

Trilostane and related compounds as defined by formula (I) or 3-enol C_{1 to 4} alkanoate esters thereof may be used in the present invention.

Preferred compounds of formula (I) are those wherein R₁ is hydrogen or methyl, R₂ is hydrogen or methyl and R₅ and R₆ are methyl. It is further preferred that R₄ is hydroxy or R₃ and R₄ together are oxo. Examples of such preferred compounds are trilostane (R₁, R₂ and R₃ are hydrogen, R₄ is hydroxy and R₅ and R₆ are methyl), ketotrilostane (R₁ and R₂ are hydrogen, R₃ and R₄ together are oxo and R₅ and R₆ are methyl) and epostane (R₁, R₃, R₅ and R₆ are methyl, R₂ is hydrogen and R₄ is hydroxy.)

The present compounds may be used in the manufacture of a medicament for the treatment of angiotensin II related disease in humans and animals. Typically, the present compounds may be used in the manufacture of a medicament for the treatment of angiotensin II related cardiovascular disease in humans and animals. Diseases which may be treated include, but are not restricted to, heart failure associated with proliferative and fibrotic changes such as congestive heart failure, post myocardial infarction, cardiomyopathy, diabetes, renal failure, metabolic syndrome (Syndrome X) and hyperaldosteronism such as primary, secondary and tertiary hyperaldosteronism and other diseases or conditions where increased levels of angiotensin II are present in the blood or the tissues of the body. A further example of an angiotensin II related cardiovascular disease which may be treated is arrhythmia. Arrhythmia and its treatment using Captopril and Losartan is discussed in Ozer et al, 2002 Pharmacol Res 45:257-63. Typically, the angiotensin II related cardiovascular disease is congestive heart failure, post myocardial infarction, cardiomyopathy, diabetes, renal failure or metabolic syndrome (Syndrome X). More typically, the angiotensin II related cardiovascular disease is post myocardial infarction.

Preferably, the angiotensin II related disease to be treated is a proliferative disease. Typically, proliferative diseases are diseases where smooth muscle cell proliferation is exhibited. Typically the proliferative disease is a cardiovascular proliferative disease. More typically, the proliferative disease is a cardiovascular proliferative disease in which angiotensin II regulated smooth muscle cell proliferation and/or smooth muscle cell migration is exhibited. Preferred examples of proliferative diseases to be treated include peripheral arterial disease, cerebro vascular disease, cardiofibrosis, cardiac myopathy, diabetic retinopathy, diabetic gangrene, diabetic nephtopathy, scleroderma, asthma, aneurism and atheroma, especially such diseases other than atheroma.

More preferably, the proliferative disease to be treated is cardiofibrosis. Yet more preferably it is cardiofibrosis following infarction. In cardiofibrosis following infarction, both the infarct size and the degree of neutrophil invasion are angiotensin II dependent. Cardiofibrosis following infarction is discussed in Sun et al, 1994 Cardiovasc Res 28:1423-32 and Waltman et al, 1995, J Card Fail 1:293-302 (infarct size and neutrophil invasion), and Wang et al, Cardiovasc Res 55:25-37 and Martinez et al 2003 Arch Med Res 34:357-61 (use of captopril and losartan in cardiofibrosis following infarction).

Typically, the patient to be treated is suffering from an angiotensin II related disease which is not associated with an increased level of adrenal steroids or an angiotensin II related disease which cannot be treated by suppressing adrenal steroid secrection.

Such compounds are preferably used in particulate form. In particular, the compounds desirably consist of particles having a mean equivalent sphere volume diameter of 12 μm or less and 80, 85, 90, 95% or more, preferably 98% or more, 99% or more or 99.5% or more of the particles have a particle diameter of less than 50 μm, preferably less than 40 μm, less than 30 μm or less than 20 μm e.g. from 0.1 μm to 10, 20, 30, 40 or 50 μm, from 1 μm to 10, 20, 30, 40 or 50 μm or from 10 μm to 20, 30, 40 or 50 μm. The particles preferably have a mean equivalent sphere volume diameter of from 5 to 12 μm or of up to 5 μm, e.g from 0.1 to 5 μm or from 1 to 5 μm. It is further preferred that the cumulative percentage oversize versus size characteristic curve of the compound of formula (I) exhibits a standard deviation of from 1.5 to 2.5 μm, preferably from 1.75 to 2.25 μm, more preferably about 2 μm, e.g. 1.9 to 2.1 μm.

The treatment is given in the form of a medicament, which preferably comprises a unit dosage of from 25 mg to 1000 mg, for example from, 25 to 50 mg, from 50 to 100 mg, from 100 to 200 mg, from 200 to 300 mg, from 300 to 400 mg, from 400 to 500 mg, from 500 to 600 mg, from 600 to 700 mg, from 700 to 800 mg, from 800 to 900 mg or from 900 to 1000 mg, of the compound of the present invention. Further examples of typical unit dosages include form 0.25 mg to 1000 mg, for example 0.5 to 25 mg, 1 to 5 mg, 5 to 10 mg, 10 to 15 mg, 15 to 20, or 20 to 25 mg.

The unit dosage described above may be administered at regular intervals such as one unit dosage administered once per month, once per week, once per day or several times per day. This treatment may be carried out for a total period of from one day, to several weeks, several months or for several years, for example for the rest of the subject's life.

It is further preferred that trilostane or related compound, as defined above, is administered in an amount of from 0.5 to 4 mg/kg/day. Most preferably, the trilostane or related compound is administered in an amount of from 1 to 3 mg/kg/day, for example from 1 to 1.5 mg/kg/day, 1.5 to 2 mg/kg/day, 2 to 2.5 mg/kg/day or from 2.5 to 3 mg/kg/day.

The compound of formula (I) or a 3-enol C_{1 to 4} alkanoate ester thereof can be present in the form of a pharmaceutically acceptable salt. As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines or heterocyclic amines.

The medicament can be administered by an intravenous, intramuscular or subcutaneous route or topically as an ointment, cream or lotion. The preferred route is oral, for instance as a tablet, a capsule or a liquid dispersion.

Whilst trilostane and the other compounds of formula (I) and esters thereof may be administered in the pure form, usually they will be formulated with one or more pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride. Solutions for injection or infusion may contain as carrier, for example, sterile water or preferably they may be in the form of sterile, aqueous, isotonic solutions.

The treatment may be used alone or in combination with a further treatment of one or more compounds from the following; an ACE inhibitor, an angiotensin II receptor blocker or an aldosterone inhibitor or agent for lowering aldosterone levels or blocking the effects of aldosterone. The aldosterone inhibitor or agent for lowering aldosterone levels may or may not be an ACE inhibitor. Examples of suitable ACE inhibitors for use in the combination treatment include, but are not restricted to, Captopril, Enalopril and Lisinopril. Suitable aldosterone inhibitors or agents for lowering aldosterone levels include, but are not restricted to, Spironolactone, Losartan and Eplerenone. Of these, Spironolactone and Eplerenone are aldosterone inhibitors and act as an antagonist at the aldosterone receptor. Losartan acts as an angiotensin II receptor blocker at the type 1 (AT1) receptor and partly exerts its physiological effect by reducing aldosterone concentrations. Another example of an angiotensin II type 1 receptor blocker is Candasartan. A further example of a compound which may be used in combination with trilostane or related compound, as defined above, is a steroidogenesis inhibitor, for example aminoglutethimide and metyrapone.

Preferably, the treatment is used alone or in combination with one or more further treatments selected from Captopril, Enalopril, Lisinopril, Spironolactone, Eplerenone, Losartan, Candasartan, aminoglutethimide and metyrapone. More preferably, the treatment is used alone or in combination with a further treatment of Losartan.

The treatment and the further treatment may be carried out simultaneously, separately or sequentially, and in either order if separate or sequential. The treatment and further treatment may be given in the form of a single combined medicament, which preferably comprises a unit dosage of said further compound in an amount known in the art to be effective in the treatment of cardiovascular disease, and a unit dosage of a compound of formula (I) or a 3-enol C_{1 to 4} alkanoate ester thereof in an amount as described above. The medicament may be administered by a mode as described above. Alternatively, the two treatments may be given separately or sequentially, e.g. as two different medicaments administered at the same site or at different sites, by the same mode of administration or by different modes of administration.

### EXAMPLES

Aortic smooth muscle cells (ASMCs) were isolated from rat thoracic and abdominal artery (RASMC) and bovine aorta (BASMC) by the media explant method and cultured over several passages.

Segments of both abdominal and thoracic aortas were obtained from rats by careful dissection from killed rats. Segments of aorta were obtained from calves under anaesthesia. The segments of aorta were placed in a depression slide containing tissue culture medium, after which the adventitia and the outer portion of each segment was carefully removed under a dissecting microscope. The remaining inner portion of the tissue and the intima were removed to a separate dissecting dish and washed several times with fresh culture medium. At this point each segment was cut into approximately 1 mm squares and placed on 25 cm² tissue culture flask. The flasks were loosely capped and placed in a humidified CO₂ incubator After two hours, 4 ml of RPMI-1640 culture medium supplemented with 100 units/ml of penicillin, 100 mg/ml streptomycin, 4 pmol/L L-glutamine and 20% PBS was carefully added to the flasks without dislodging the tissue. Samples were fed with fresh medium after one week. The cells from the explants were relatively confluent within a period of approximately 2 weeks. They were then rinsed with PBS, and subsequently trypsinized with a solution of 0.125% trypsin and 0.02% EDTA in PBS for 1-2 min al 37° C. The resulting suspension of cells was pipetted into 75 cm² tissue culture flasks containing 10 ml culture medium and incubated as above.

Experiments were performed with cells from passages 3 to 5.

### Example 1

³H-methylthymidine incorporation into rat aortic smooth muscle cells (RASMC). Quiescent RASMC (0.3×10⁵/ml/well) were incubated with serum-free medium (SFM) containing Ang II (10⁻⁷ M) with or without different concentrations of trilostane for 48 hours. The results are shown in Table 1. ³H-methylthymidine incorporation into RASMC was increased in the Ang II treated group. The tritium incorporation induced by Ang II was inhibited by trilostane at 10⁻⁶ and 10⁻⁵ but not at 10⁻⁹, 10⁻⁸ and 10⁻⁷ M.

TABLE 1

| | | Concentration of | Tritiated |
|---|---|---|---|
| Sample | Concentration of Ang II | Trilostane added to | thymidine |
| No. | added to sample (M) | sample (M) | uptake (dpm) |
| 1 | — (control) | — (control) | 57.6 |
| 2 | 10⁻⁷ | — | 87.1 |
| 3 | 10⁻⁷ | 10⁻⁹ | 96.7 |
| 4 | 10⁻⁷ | 10⁻⁸ | 101.88 |
| 5 | 10⁻⁷ | 10⁻⁷ | 89.1 |
| 6 | 10⁻⁷ | 10⁻⁶ | 74.9 |
| 7 | 10⁻⁷ | 10⁻⁵ | 42.7 |

Values are means ± S.E.M. N = 3 per group. ANNOVA: P < 0.001; Student's t-test - Comparison of controls with angiotensin simulated, P < 0.01, Comparison of angiotensin stimulated with trilostane added at 10⁻⁶ or 10⁻⁵ M, P < 0.05. (dpm: disintergrations per minute)

### Example 2

Cell count for rat aortic smooth muscle cells (RASMC). RASMC (0.5×10⁵/ml/well) were incubated with 20% FBS RPMI-1640 medium containing Ang II (10⁻⁷ M) with or without different concentrations of trilostane for 48 hours. The results are shown in Table 2. Number of RASMC in groups treated with Ang II 10⁻⁷ M was significantly increased, compared with controls. The Ang II stimulated increase in cell number was inhibited by trilostane at 10⁻⁶ and 10⁻⁵ but not at 10⁻⁹, 10⁻⁸ and 10⁻⁷ M.

TABLE 2

| | | Concentration of | |
|---|---|---|---|
| Sample | Concentration of Ang II | Trilostane added to | |
| No. | added to sample (M) | sample (M) | Cell Count |
| 8 | — (control) | — (control) | 12.00 × 10⁴ |
| 9 | 10⁻⁷ | — | 21.30 × 10⁴ |
| 10 | 10⁻⁷ | 10⁻⁹ | 21.00 × 10⁴ |
| 11 | 10⁻⁷ | 10⁻⁸ | 21.95 × 10⁴ |
| 12 | 10⁻⁷ | 10⁻⁷ | 20.00 × 10⁴ |
| 13 | 10⁻⁷ | 10⁻⁶ | 14.00 × 10⁴ |
| 14 | 10⁻⁷ | 10⁻⁵ | 14.25 × 10⁴ |

Values are means ± S.E.M. N = 3 per group. ANOVA: P < 0.001; Student's t-test- Comparison of controls with angiotensin stimulated, P < 0.01, Comparison of angiotensin stimulated with trilostane added at 10⁻⁶ or 10⁻⁵ M, P < 0.05.

### Example 3

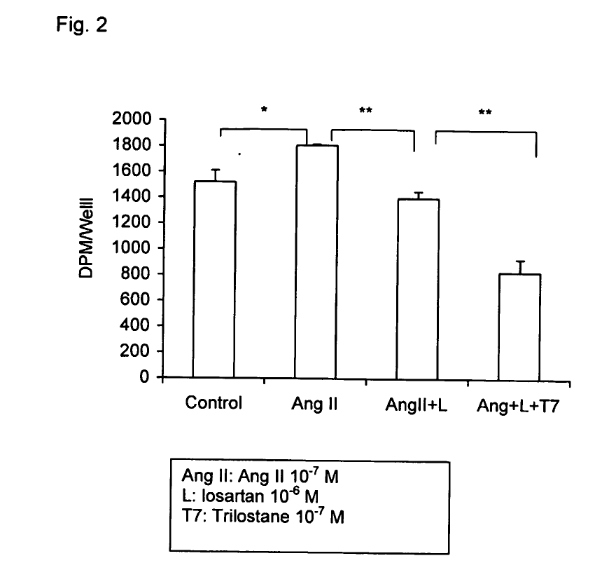

Using the same tritiated thymidine uptake methodology as for Example 1, the actions of Losartan on angiotensin II-stimulated cell proliferation were tested in the presence and absence of trilostane. Losartan significantly eliminated the stimulatory action of angiotensin II, and the losartan alone group were not different from controls. The additional presence of trilostane decreased cell proliferation still further to less than control values (*P<0.05, **P<0.01). The results are shown in FIG. 2.

### Example 4

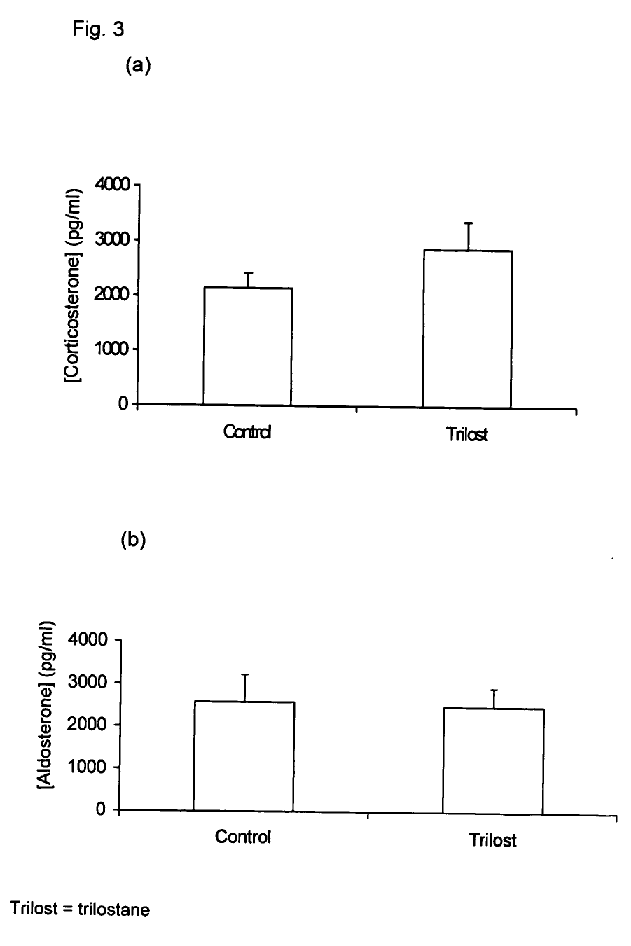

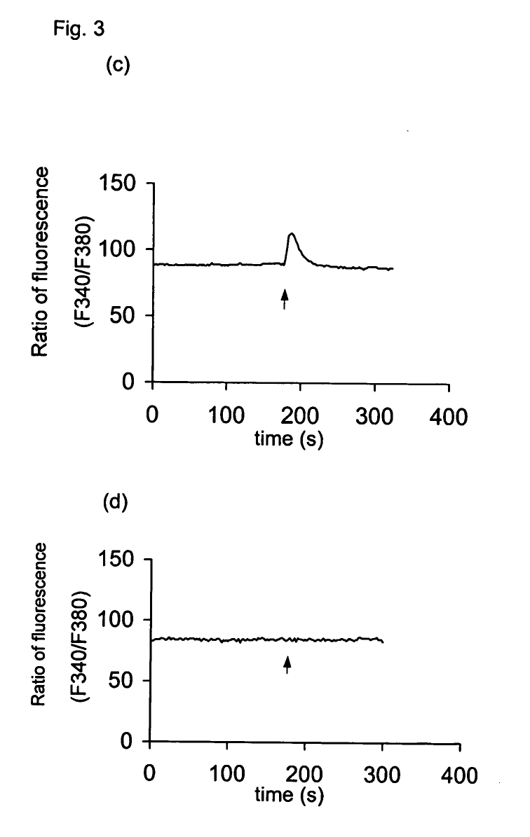

Male Wistar rats (˜500 g) were treated with 0.1 ml ethanol in cotton seed oil with trilostane 4 mg/kg per day for 5 days. Control animals received the vehicle alone. Animals were then treated with 0.1 ml (500 u) heparin IP, before killing by stunning and cervical dislocation. Blood was collected from neck vessels, centrifuged to obtain plasma which was stored at −20° C. until required for steroid analysis. Corticosterone and aldosterone concentrations were assayed using commercially available kits (Diagnostic Systems Laboratories Inc., Webster, Tex., USA). The results are shown in FIG. 3. Circulating concentrations of corticosterone (FIG. 3 (a)) and aldosterone (FIG. 3(b)) are shown for control animals and animals receiving trilostane treatment. There are no significant differences between control and trilostane values.

Primary cultures were established of RASMC obtained from trilostane treated and control animals, using the established methods. For [Ca²⁺]ᵢ measurement, the cells were loaded with 1 μM fura-2 for 30 min in medium-modified Krebs-Ringer bicarbonate solution (3.6 mM K⁺, 1.2 mM Ca²⁺, 0.5 mM Mg²⁺, 5 mM Hepes and 20 mM HCO⁻) at 37° C. For simultaneous measurements of measuring the fluorescence of fura-2, the cells plated on coverslips were mounted on the stage of an inverted microscope (Zeiss) in a modified Krebs-Ringer bicarbonate solution. The excitation wavelengths were 340 and 380 nm, and emission was detected at 510 nm. [Ca²⁺]ᵢ was calculated from the ratio of fluorescence intensities at excitation wavelengths of 340 and 380 nm. Fields of cells, ˜10 cells per field, were tested from control and trilostane treated animals. The results are shown in Table 3 and FIGS. (c) and (d) wherein the arrow indicates the time of application of angiotensin II. Characteristic calcium signals obtained by stimulation of vascular smooth muscle cells from control animals (FIG. 3(c)) and trilostane treated animals (FIG. 3(d)) by 10 nmol/L angiotensin II.

TABLE 3
Threshold concentrations for calcium signal responses
to angiotensin II (1 nmol/L) in trilostane treated smooth
muscle cells (TTSMC) and control cells (NSMC).

Ang II (M)

| Cells | 10⁻¹¹ | 10⁻¹⁰ | 10⁻⁹ | 10⁻⁸ | 10⁻⁷ | 10⁻⁶ |
|---|---|---|---|---|---|---|
| TTSMC | − | − | − | − | − | + |
| NSMC | − | − | − | + | | |

+ = calcium response, − = no response

### Example 5

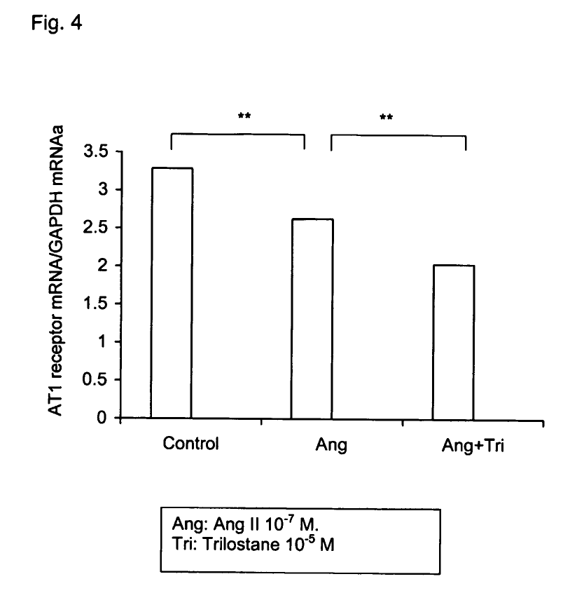

The expression of AT1 receptor mRNA was detected by RT-PCR and real-time quantitative RT-PCR in RASMC incubated with or without aldosterone 10⁻⁸ mol/L for 48 hours. Real-time RT-PCR was performed using Brilliant SYBR Green QRT-PCR Master Mix Kit, 1-step based on real-time detection of accumulated fluorescence (M×300P, Stratagene, Amsterdam). Results are means, SEM too small to show. **=P<0.01. The results are shown in FIG. 4. Angiotensin II itself reduces mRNA transcription of the gene coding for the angiotensin II type 1 receptor (AT1), and this is reduced even further by addition of trilostane.

What is claimed is:

1. A method of treating an angiotensin II related disease in a patient, comprising administering to a patient in need thereof an effective amount of a compound of formula (I): 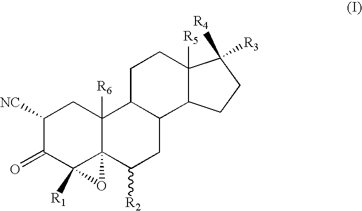
or a 3-enol C_{1 to 4} alkanoate ester thereof,
wherein R₁, R₂, R₅, R₆ are the same or different and each is hydrogen or C_{1 to 4} alkyl;
R₃ is hydrogen, C_{1 to 4} alkyl, C_{2 to 4} alkenyl or C_{2 to 4} alkynyl;
R₄ is hydroxyl, C_{1 to 4} alkanoyloxy, or a group of formula (II) or (III) 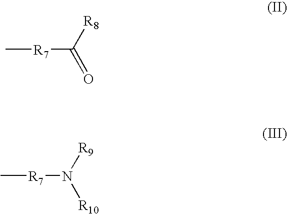
wherein R₇ is (CH₂)ₙ, where n is an integer of from 0 to 4, R₈ is hydrogen, C_{1 to 4} alkyl, hydroxy or NH₂ and R₉ and R₁₀ are the same or different and each is hydrogen or C_{1 to 4} alkyl;
or R₃ and R₄ together are oxo, ethylenedioxy or propylenedioxy.

2. A method according to claim 1, wherein in formula (I) R₁ is hydrogen or methyl; R₂ is hydrogen or methyl; R₄ is hydroxy or R₃ and R₄ together are oxo; and R₅ and R₆ are methyl.

3. A method according to claim 1, wherein the compound of formula (I) is trilostane, ketotrilostane or epostane.

4. A method according to claim 1, wherein the angiotensin II related disease is a cardiovascular disease.

5. A method according to claim 4, wherein the cardiovascular disease is congestive heart failure, post myocardial infarction, cardiomyopathy, diabetes, renal failure, metabolic syndrome (Syndrome X) or arrhythmia.

6. A method according to claim 4, wherein the cardiovascular disease is post myocardial infarction.

7. A method according to claim 1, wherein the compound of formula (I) is administered in an amount of from 0.5 to 4 mg/kg/day.

8. A method according to claim 1, wherein the angiotensin II related disease is a proliferative disease.

9. A method according to claim 8, wherein the proliferative disease is peripheral arterial disease, cerebro vascular disease, cardiofibrosis, cardiac myopathy, diabetic retinopathy, diabetic gangrene, diabetic nephropathy, scleroderma, aneurism, asthma or atheroma.

10. A method according to claim 9, wherein the proliferative disease is cardiofibrosis.

11. A method according to claim 8, wherein the proliferative disease is cardiofibrosis following infarction.

12. A method according to claim 8, wherein the compound of formula (I) is administered in an amount of from 0.5 to 4 mg/kg/day.

13. A method according to claim 1 wherein the compound of formula (I) is in particulate form.

14. A method according to claim 13 wherein the particles of the particulate form compound have a mean equivalent sphere volume diameter of up to 12 μm and 95% or more of the particles have a particle size of up to 50 μm.

15. A method according to claim 14 wherein the particles have a mean equivalent sphere volume diameter of from 5 to 12 μm.

16. A method according to claim 13 wherein the particles have a mean equivalent sphere volume diameter of up to 5 μm.

17. A method according to claim 13 wherein the specific surface area of the particulate compound is 2 m² g⁻¹ or higher or 5 m² g⁻¹ or higher.

18. A method according to claim 1 wherein the compound of formula (I) is administered orally either as a tablet, a capsule or a liquid dispersion.

19. A method according to claim 1 comprising administering a unit dosage of from 0.25 mg to 1000 mg of a compound of formula (I) or a 3-enol C_{1 to 4} alkanoate ester thereof.

20. A method according to claim 19 wherein the unit dosage is from 0.5 mg to 25 mg.

21. A method according to claim 19, wherein the unit dosage is from 25 to 1000 mg.

22. A method according to claim 1 wherein the treatment of an angiotensin II related cardiovascular disease with a compound of formula (I) or a 3-enol C_{1 to 4} alkanoate ester thereof is carried out in combination with a further treatment of one or more of:
an Angiotensin Converting Enzyme (ACE) inhibitor;
an angiotensin II receptor blocker;
an aldosterone inhibitor or agent used for lowering aldosterone levels or blocking the effects of aldosterone in the body; or
a steroidogenesis inhibitor.

23. A method according to claim 22 wherein the aldosterone inhibitor or agent used for lowering aldosterone levels is an ACE inhibitor.

24. A method according to claim 23 wherein the ACE inhibitor is Captopril, Enalopril or Lisinopril.

25. A method according to claim 22 wherein the aldosterone inhibitor or agent for blocking the effects of aldosterone is Spironolactone or Eplerenone.

26. A method according to claim 22 wherein the angiotensin II receptor blocker is Losartan or Candasartan.

27. A method according to claim 22 wherein the steroidogenesis inhibitor is aminoglutethimide or metyrapone.

28. A pharmaceutical composition comprising:
(a) a compound of formula (I) or a 3 -enol C_{1 to 4} alkanoate ester thereof as defined in claim 1; and
(b) one or more of:
an ACE inhibitor;
an angiotensin II receptor blocker;
an inhibitor or agent used for lowering aldosterone levels or blocking the effects of aldosterone; or
a steroidogenesis inhibitor.

29. (canceled)

30. A method of treating an angiotensin II related disease by administering to a patient having said disease an amount of a compound of formula (I) or a 3-enol C₁ₜₒ₄ alkanoate ester thereof as defined in claim 1 and an amount of one or more of:
an ACE inhibitor;
an angiotensin II receptor blocker;
an aldosterone inhibitor or agent for lowering aldosterone levels or blocking the effects of aldosterone; or
a steroidogenesis inhibitor
effective to treat said disease.

31. A method according to claim 30 wherein the angiotensin II related disease is a cardiovascular disease or a proliferative disease.
